(19) United States
(12) Patent Application Publication
(10) Pub. No.: US 2013/0103046 A1
(43) Pub. Date: Apr. 25, 2013

(54) ROTARY CATHETER FOR REMOVING OBSTRUCTIONS FROM BODILY VESSELS

(71) Applicant: Samuel Shiber, Santa Barbara, CA (US)

(72) Inventor: Samuel Shiber, Santa Barbara, CA (US)

(21) Appl. No.: 13/695,232

(22) PCT Filed: Apr. 5, 2011
(86) PCT No.: PCT/US2011/031197

§ 371 Date: Jan. 8, 2013

Related U.S. Application Data
(60) Provisional application No. 61/343,796, filed on May 4, 2010.
(60) Provisional application No. 61/461,263, filed on Jan. 14, 2011.

(51) Int. Cl. A61B 17/22

(52) U.S. Cl. CPC A61B 17/22
USPC 606/127

(57) ABSTRACT

A motorized rotary catheter, for breaking down and aspirating an obstruction from within a bodily vessel comprising a flexible tube containing a motor-driven, flexible, hollow shaft that is rotatable and slidable over a guidewire; a distal portion of the shaft is made of a closely wound spiraled wire and a distal portion of that spiraled wire is extendable out of a distal end of the tube; a bit affixed to a distal end of the spiraled wire forms therewith a tip that has first and second opposing sides for impacting and breaking down the obstruction as the shaft rotates; the tip also has a base and an opposing crown that is offset away from a longitudinal axis of the shaft further than the base is, enlarging the area that the tip sweeps as it rotates; a distance between the sides being smaller than a distance between the crown and the base, enhancing the ability of the tip to pass through tight spots along the vessel; as the tip rotates and the crown atraumatically slides against a wall of the vessel it displaces the distal end of the tip away from the wall to prevent the distal end of the tip from starting to tunnel through the obstruction too close to the wall.

### BACKGROUND AND SUMMARY OF THE INVENTION

Pharmacological, surgical and current trans-catheter treatments of vascular obstructions can be time-consuming, traumatic and expensive and it is an object of the present invention to simplify, improve and shorten the procedure by enabling the physician to readily navigate and advance the rotary catheter through curved vessels and bifurcations to the obstruction site and to break the obstruction into particles that can be removed by a mechanically enabled aspiration.

One embodiment, according to the present invention, comprises a flexible tube containing a motor-driven, flexible, hollow shaft that is rotatable and slideable over a guidewire. At least a distal portion of the shaft is made of a closely wound spiraled wire with a bit affixed to its distal end to form therewith a tip that is rotatable inside or outside the tube. The tip has a first side and an opposing second side. The first side is adapted to impact the obstruction when the shaft is rotated in a first direction and the second side is adapted to impact the obstruction when the shaft is rotated in a second direction.

The tip also has a base and an opposing crown that is offset away from a longitudinal axis of the shaft further than the base, enlarging an area that the tip sweeps when rotating outside of the tube. The distance between the sides is smaller than the distance between the crown and the base, leaving open aspiration passageways through the tube when the tip is inside the tube and also enhancing the tip's ability to pass through tight spots along the vessel. The crown is adapted to atraumatically slide against the vessel's wall and displace a distal end of the tip away from the wall while the tip rotates.

The rotary catheter can be inserted into the vessel directly (e.g., when access to the vessel is gained surgically) or through the skin via an introducer. The introducer's side arm can be used to inject fluids into the vessel, e.g., a mixture of saline, heparin and a radio-opaque contrast agent, or alternatively, to aspirate fluids and particles from the vessel.

An optional guiding catheter can be used to guide the rotary catheter further into the vessel. The guiding catheter can incorporate a proximal embolic barrier for temporarily blocking flow through the vessel to allow the rotary catheter to macerate and aspirate the obstructing material while reducing the chance of releasing particles downstream. A distal embolic protection device can also be employed for same purpose and, where the rotary catheter is used in a limb, an external pressure cuff can be utilized for the same purpose.

A passageway, defined through the rotary catheter housing, connects the flexible tube with an external port so that the port can be utilized to aspirate fluids and particles from the vessel.

To prevent the flexible tube from kinking (i.e., diametrically collapsing) and to prevent the shaft from being sharply bent at the point in which they are connected to the housing, their radius of bending is limited to a radius of curvature of a wall of a depression defined by the housing area that surrounds the tube.

The rotary catheter can be manufactured in varying lengths and diameters to reach and treat different locations in the human anatomy and different forms of occlusive diseases, as well as to suit variations in the methods and preferences of the user.

### BRIEF DESCRIPTION OF THE DRAWINGS

FIG. 1 shows an overview of a motorized rotary catheter, according to the present invention, with a tip and a distal portion of a spiraled wire extending out of a distal end of a flexible tube (“distal” refers to a location or direction further into the vessel and “proximal” means the reverse);

FIG. 1a shows an overview of the rotary catheter with a distal end of its flexible tube moved nearer to the tip;

FIG. 1b shows enlargement of a rotary seal area;

FIG. 2 shows an enlargement of a region marked 2 on FIG. 1;

FIG. 3 shows a front view of the tip as viewed on a plane 3-3 marked on FIG. 2;

FIGS. 4, 5 and 6 show examples of flattened cross section of a wire that can be used to wind a spiraled wire;

FIG. 7 shows an enlargement of a region marked 7 on FIG. 1 where an optional welded connection of shaft's portions is shown;

FIG. 8 shows an enlargement of a region marked 8 on FIG. 1;

FIG. 9 shows an enlargement of a region marked 9 on FIG. 1;

FIGS. 10 and 10a shows an overview of a modified rotary catheter wherein the tube can be selectively moved distally over the tip to shield it as shown in FIG. 10a,

FIG. 10b shows an end view of the rotary catheter as viewed on a plane 10b-10b marked on FIG. 10a;

FIG. 11 shows a further modification of the rotary catheter wherein the distal end of the tube is terminated diagonally;

FIG. 12 shows a further modification of the rotary catheter wherein the shape of the distal end of the tube resembles a scoop of a garden trowel;

FIG. 13 shows a further modification of the rotary catheter wherein the sheath resembles a scoop of a garden trowel with a thickened bottom;

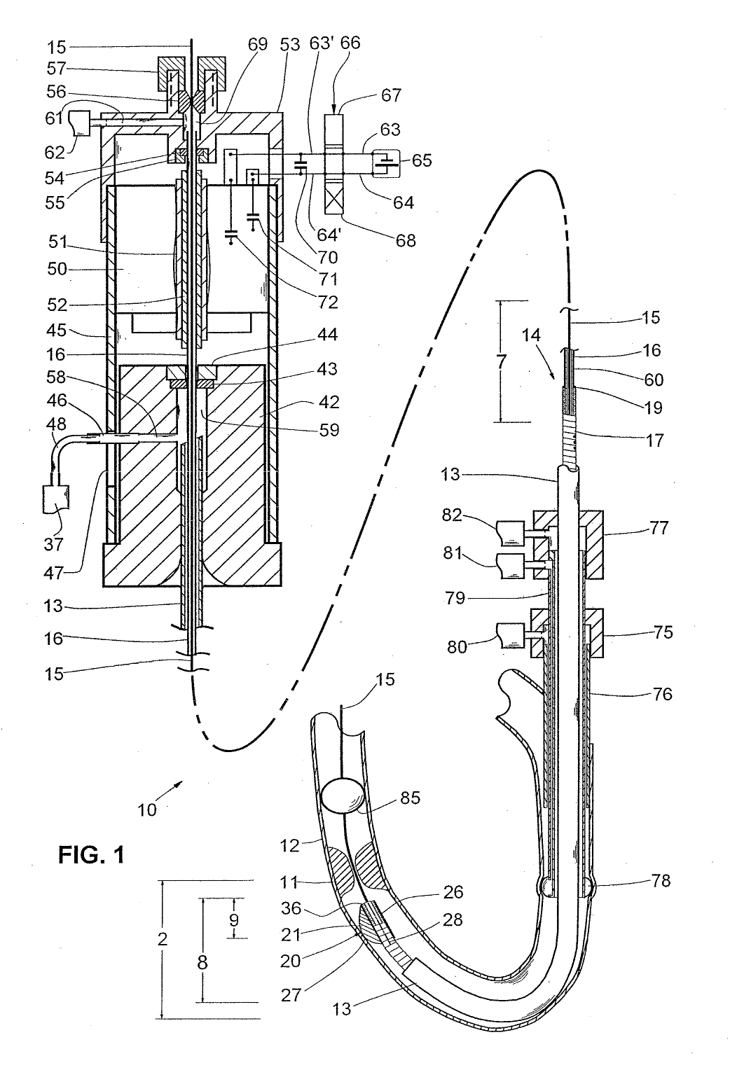

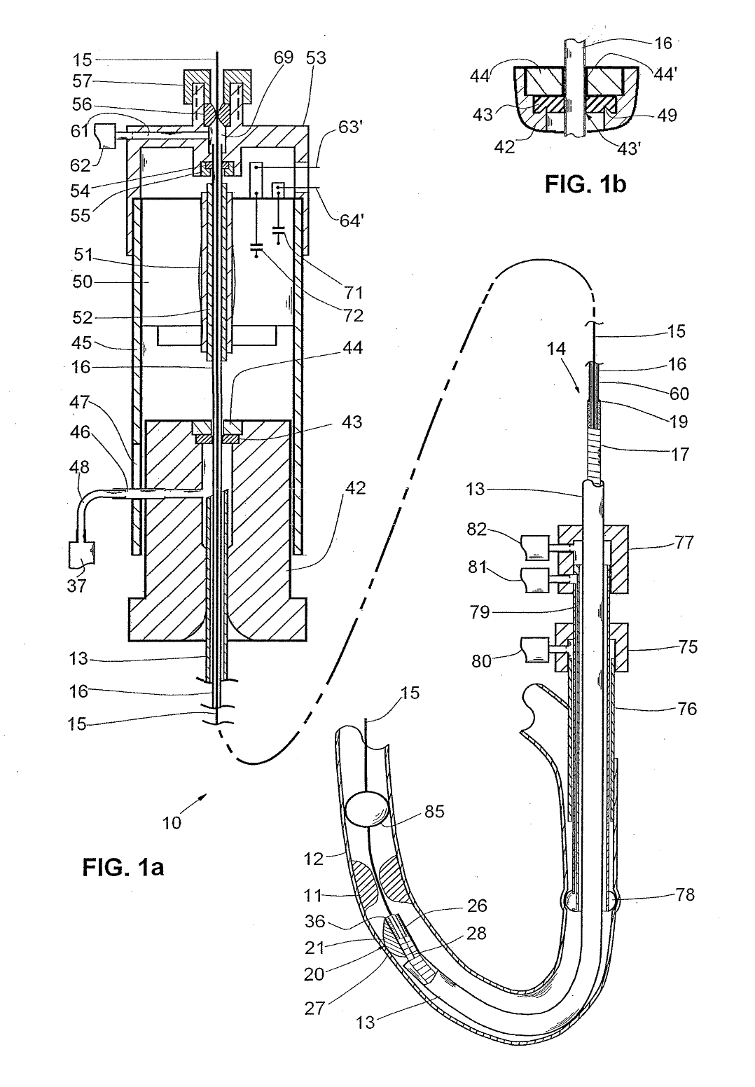

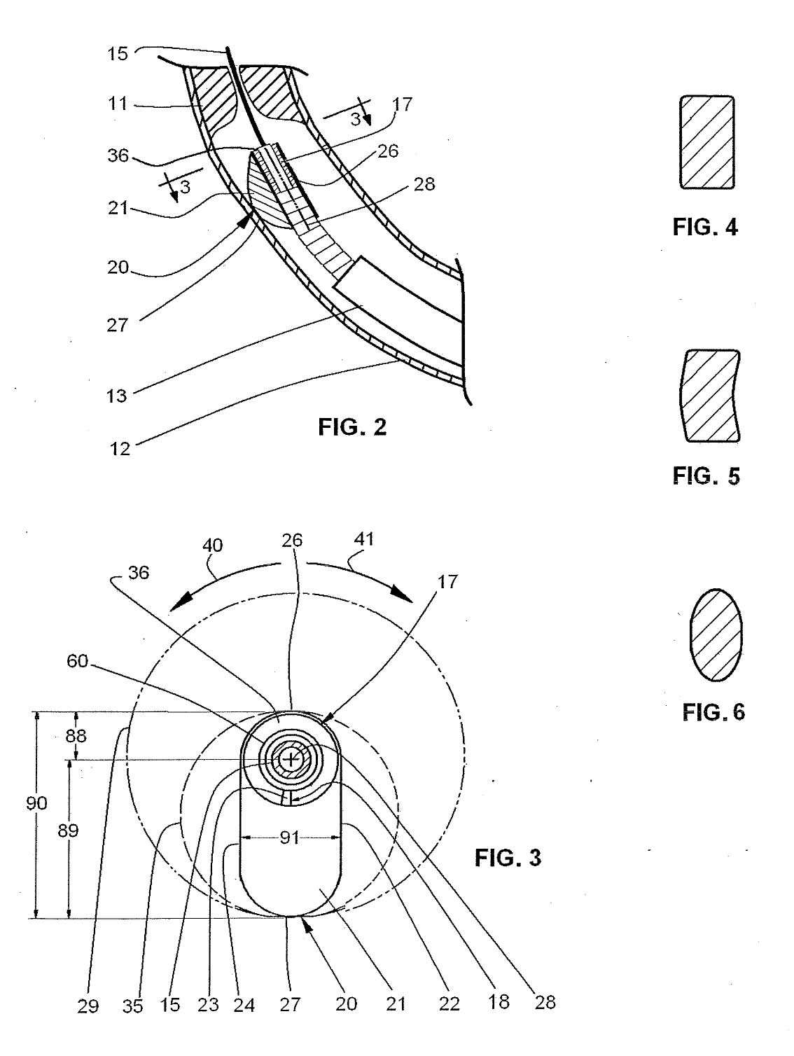

The middle portion of the embodiments shown in FIGS. 1, 1a, 10, 10a, 11, and 12 is represented by a phantom line due to space limitations on the drawing sheet.

### DETAILED DESCRIPTION OF THE EMBODIMENTS

FIG. 1 show a motorized rotary catheter 10, according to the present invention, for breaking down and for removing an obstruction 11 (e.g., thrombus, atheroma) from within a bodily vessel 12 (e.g., a blood vessel.)

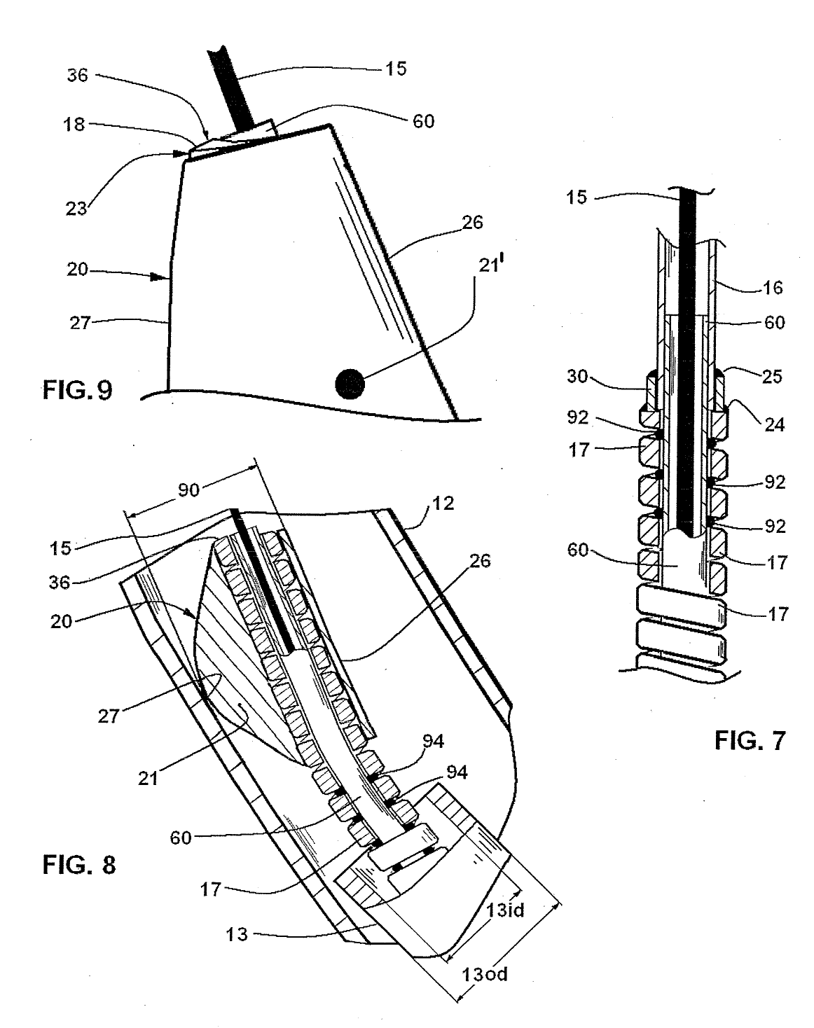

The rotary catheter 10 comprises a flexible tube 13, preferably made of a thin plastic, which contains a motor-driven, flexible, hollow shaft 14 that is rotatable and slideable over a guidewire 15. A proximal portion 16 of the shaft is a thin-walled tube and a distal portion of the shaft 17 is preferably made of a closely wound spiraled metal wire. The portions 16 and 17 are connected together, for example, by a circumferential weld 19 (please note FIG. 1) or alternatively by two circumferential welds 24 and 25 and a short reinforcing sleeve 30 (please note FIG. 7.) The portions 16 and 17 are preferably made of metal, for example stainless steel or Nitinol, and the wire used to wind the spiraled wire preferably has a flattened cross-section (please note FIGS. 4-6.) Such a cross section can be obtained by taking a standard round wire and running it between rollers that squeeze and flatten it.

A bit 21 is affixed by a weld 21′ to a distal end of the spiraled wire to form therewith a tip 20 (please note FIGS. 9 and 8). The weld is at point along the spiral that is nested inside the tip where it subjected mostly to shearing loads and is otherwise protected. The tip has a first side 22 for impacting the obstruction as the shaft is rotated in a first direction 40 and an opposing second side 24 for impacting the obstruction when the shaft is rotated in a second direction 41 (please note FIG. 3.)

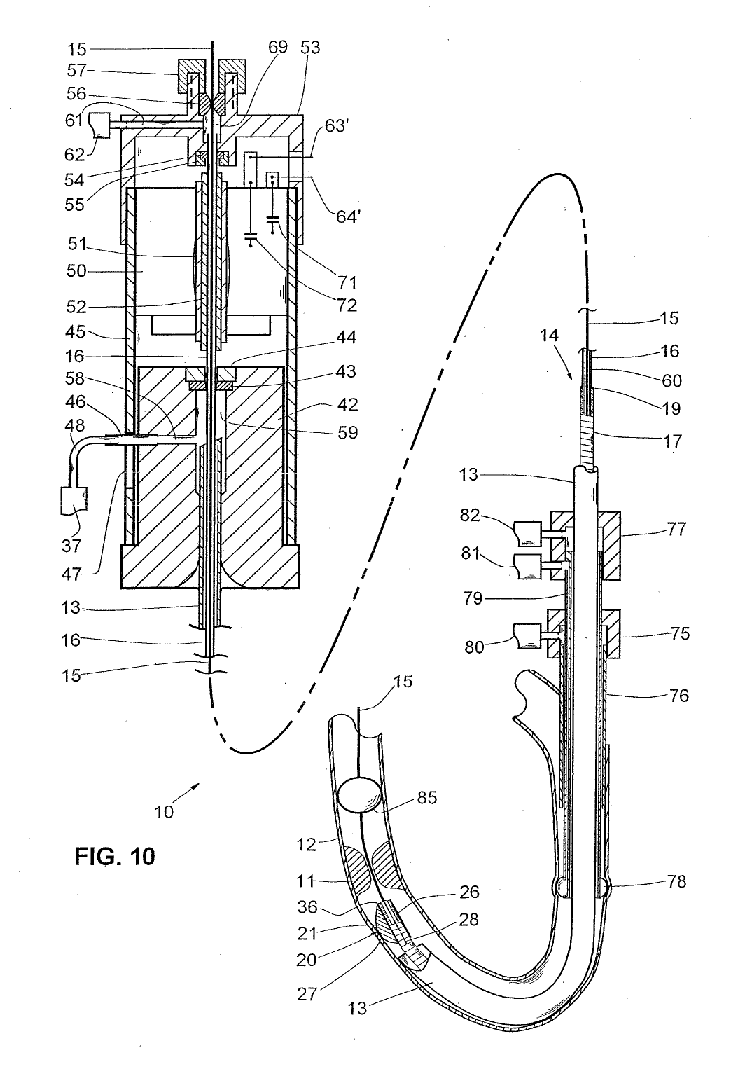

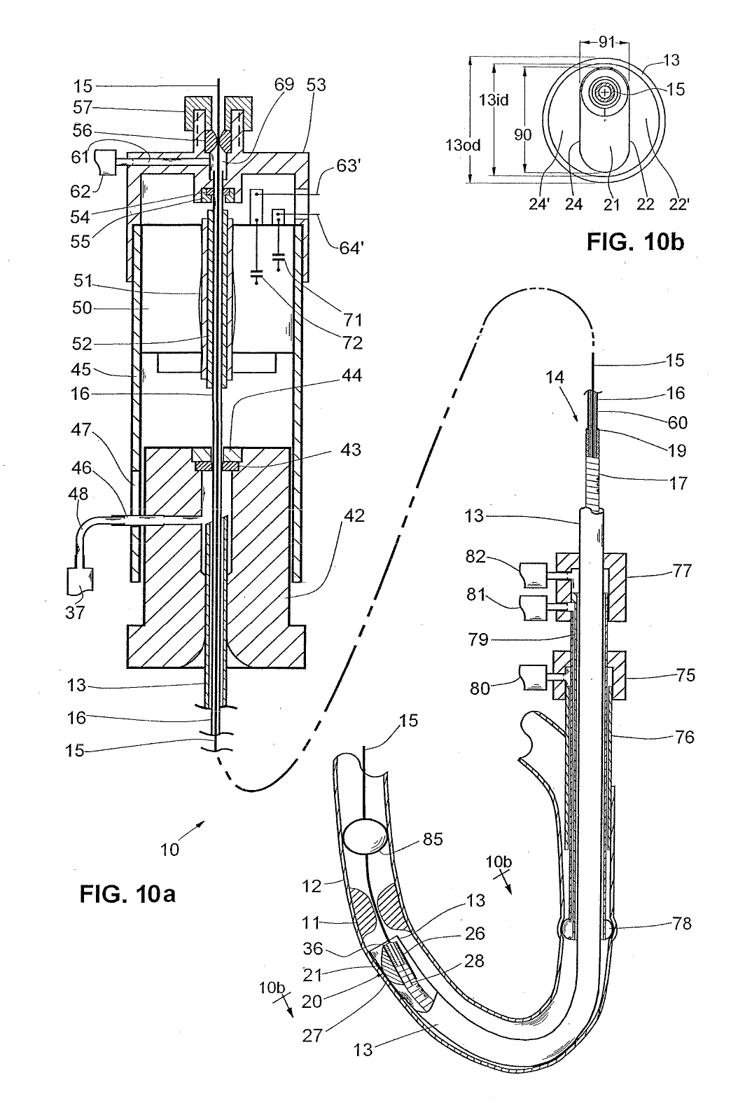

The tip also has a base 26 and an opposing crown 27. The crown is offset (the term offset refers to a distance from a longitudinal axis 28 of the spiraled wire 17) by a distance 89 which is larger than an offset of the base 88. The distance 89 is also the nominal radial reach of the crown while it rotates around the axis 28 (please note FIG. 3.) As the tip rotates around axis 28 and the crown slides along a phantom line 29, the tip sweeps an enlarged area within a line 29. This swept area within line 29 is substantially larger than an area within an interrupted line 35 that a hypothetical symmetrically mounted tip (i.e. 88 equals 89) would have swept. However, it should be understood that the actual swept area may be affected by variation in the trajectory of the crown due to the vessel's geometry, to forces that develop between the tip and its surroundings as well as by dynamic forces due to the tip's rotation (e.g., centrifugal force.) The tip has a flattened cross section where a distance 91 between its sides 22 and 24 is smaller than the distance 90 between the crown 27 and the base 26. This reduces the cross-sectional area and circumference of the tip 20 and the corresponding size of a puncture needed to insert the tip into the vessel and it enhances the tip's ability to pass through tight spots along the vessel, like a key with a flat bit passing through a narrow keyhole, as compared to a hypothetical tip with a round cross section that would be defined by the line 35. The flat cross section also enables the rotary catheter to aspirate fluid and obstruction particles around the tip 20 while the tip is inside the tube 13 (please note FIG. 10b.) As the particles pass through passageways between the tip 20 and the tube 13 they become macerated and are readily aspirated through the tube into a syringe 37 as discussed below.

Referring to FIG. 1 the tube 13 is affixed (e.g., bonded) to a cylinder 42 that also houses a seal 43. The outer periphery of the seal is tightly pressed by a bushing 44 against a circular ridge 49 forming a peripheral static seal (the ridge is shown in the enlarged view, please note FIG. 1b.) A bore 44′ in the bushing acts as a bearing which forces the shaft portion 16 that is sufficiently flexible for its proximal end to be offset and to rotate concentrically relative to a bore 43′ that is formed through the seal 43. This forced concentricity on the one hand nulls the effect of the cumulative eccentricities contributed by parts numbers 42, 45, 50, 51, 52 and shaft portion 16, reducing the interference fit needed between the bore 43′ and the shaft portion 16 to maintain a rotary seal between them and thereby it reduces frictional power loss in the seal. On the other hand it eases the tolerances that the parts 42, 45, 50, 51, 52 and shaft portion 16 have to be manufactured to and thereby it lowers the manufacturing costs of the embodiment.

The cylinder 42 is slidingly disposed in a distal end of a tubular housing 45 and a ferrule 46, that is press-fitted into the cylinder 42, is slidingly disposed in an elongated slot 47 defined in the housing 45. This allows the cylinder 42 to slide proximally into the housing (as shown in FIG. 1) or to slide distally (as shown in FIG. 1a) pulling or pushing the tube 13 over the shaft 14 to expose or to shield the distal end of the shaft, respectively. This in turn increases or decreases the flexibility of the distal end of the catheter, respectively, and also enables the user to affect and optimize the aspiration through the tube 13.

A flexible tube 48, the ferrule 46, bores 58 and 59, and seal 43 define together a hydraulic connection between the tube 13 and a suction means in the form of the evacuated syringe 37 for aspirating fluid and particles of the obstruction (only the front end of the syringe is depicted however syringes and vacuum syringes are commercially available from, for example, Merit Medical Systems, South Jordan, Utah.) The relative motion between the tube 13 and the rotating shaft 14 assists with the aspiration by reducing the frictional resistance that these particles encounter while moving proximally within the tube 13. Both tubes 13 and 48 are preferably transparent to allow the user to visually verify the rate of aspiration and to re-evacuate the syringe 37 as needed.

A DC motor 50 is housed in a proximal end of the housing 45, however other types of electric or air-driven motors, and the like, can be used. The motor has a tubular metal output shaft 51 through which the proximal end of the shaft portion 16 passes. A plastic sleeve 52 power transmittingly couples the shaft 51 and the shaft portion 16, while at the same time electrically insulating one from the other.

The shaft portion 16 is shown connected and bonded to an optional flexible guidewire-liner 60 that is preferably made of a thin-walled plastic tube (please note FIG. 7.) Optionally, a short string 92 is wrapped around and bonded through the gap between several proximal coils of the spiral wire 17 to secure the liner 60 to the spiral wire. FIG. 8 is an enlarged cross-section of a distal end of the spiral wire 17 and of the tip 20 where the distal end of the liner 60 is bonded to the spiral wire and is similarly optionally secured to it by a short string 94.

Referring back to FIG. 1, a proximal cap 53 houses a seal 54, which seals around the shaft portion 16, and is peripherally secured in place by a bushing 55, which like the bushing 44, also serves as a bearing that keeps the shaft portion 16 rotating concentrically relative to the seal 54 with the beneficial effects discussed in connection with the bushing 44. The cap 53 also houses a seal 56 that closes and opens around the guidewire 15 in response to being squeezed or un-squeezed as a threaded collar 57 is turned clockwise or counterclockwise, respectively. A syringe 62 is hydraulically connected through a passage 61 and a bore 69 defined in the cap 53 and by the seals 54 and 56, to a proximal end the shaft portion 16. The syringe 62 can be used to introduce fluid mixture (e.g., a mixture of saline and heparin) into the shaft portion 16 and into the liner 60 to prevent blood from entering and clotting in the liner and in the shaft portion 16.

Optionally only the distal portion of the liner 60 is used, to reduce blood flow into the tip, while the proximal portion of the liner is omitted. In such a case the passage 61 can be used to convey irrigating fluid to keep the volume between seals 54 and 56 and the proximal end of shaft portion 16 immersed in order to prevent air from entering into it if the seal 56 is inadvertently opened. To supply such irrigating fluid syringe 62 is preferably replaced with an elevated saline bag (not shown) that contains the irrigating fluid and feeds it to the passage 61 under gravity.

Electrical wires 63, 63′, 64 and 64′ connect the motor 50 to a battery 65 through a sliding four position switch 66. In the position shown in FIG. 1 wire 63 is connected to wire 63′ and wire 64 is connected to wire 64′ causing the motor to rotate in the first direction. When block 68 is moved upwards the wires are crossed so that wire 63 is connected to wire 64′ and wire 64 is connected wire 63′ causing the motor to rotate in the second direction and manually alternating between these positions will cause the motor to rotate back and forth. When the switch is slid downwards an electronic circuit contained in a block 67 is interposed between the wires 63 and 64 to the wires 63′ and 64′ and automatically causes the motor to rotate back and forth (the electronic circuit not shown but is familiar to the artisan.) In a fourth off-position (not shown) the switch disconnects the battery from the motor.

To reduce electromagnetic emissions from the motor a disk varistor, available from TDK Corp., Uniondale, NY., can be installed inside the motor and capacitors 70, 71 and 72 can be connected to the motor and wiring as shown. Alternatively, a 3-way capacitor, available from Johanson Dielectrics in Sylmar, CA, can be connected to wires 63 and 64 and the housing. Ferrite beads (not shown) can also be disposed along the wires 63, 64 and 63′, 64′ to further reduce electromagnetic emissions that originate in the motor.

A syringe 80 is connected through an introducer 75 to the vessel and can be used for the introduction of a fluid mixture (e.g., a mixture of saline, heparin, a radio-opaque agent and antispasmodic medication) into the vessel to make up for the volume that is aspirated through the rotary catheter and to prevent blood from entering the introducer and clotting therein. Alternatively the syringe 80 can be used to withdraw fluid and particles out of the vessel especially while the rotary catheter 10 is not disposed in the introducer. In cases where the target obstruction 11 is distant from the puncture site, a conventional guiding catheter (not shown) is optionally disposed in the introducer, to guide the rotary catheter 10 to the obstruction. Alternatively a specialized guiding catheter 77 with a toroidal shaped balloon 78 can be used to also seal flow through the vessel and reduce the likelihood of escapement of particles into the blood stream. The balloon 78 is inflatable/deflatable through a channel 79, defined in a wall of the guiding catheter, with a syringe 81 that is connected to the channel 79. A syringe 82 can be used to inject fluid mixture through the guiding catheter into the vessel to make up for the volume that is aspirated through the rotary catheter and to prevent blood from entering the guiding catheter and clotting therein. However, syringe 82 can also be used to aspirate fluid and particles out of the vessel especially while the rotary catheter 10 is not disposed in it. It can be noted that syringe 82 or syringe 80 can be replaced with a bag containing a fluid mixture under pressure, which is preferably slightly higher than the patient's blood pressure, to automatically infuse the fluid mixture and replace the volume of blood and particles that were aspirated into the syringe 37. While syringes 62, 80, 81 and 82 are illustrated as being connected directly to various other components it is understood that they can be connected through flexible tubes similar to flexible tube 48.

The guidewire 15 can be a conventional guidewire or it can be equipped with a distal particle barrier such as a filter (not shown) or a balloon 85 that is selectively inflatable through the guidewire 15.

Blood vessels, and other bodily vessels, tend to be curved and bias the rotating tip towards the wall of the vessel (please note FIG. 1.) Absent a correction, such bias tends to lead the tip to begin tunneling into the obstruction adjacent to the wall, especially in a case of an obstruction that totally blocks the vessel. However, with the present invention, as the rotating crown 27 slides against the wall of the vessel, it displaces a distal end 36 of the tip away from the wall (please note FIG. 8.) This correction mechanism urges the distal end of the tip to start tunneling away from the wall. It can be understood by the artisan that this correction mechanism would not work if the flexible spiraled wire would have extended distally beyond the bit where the bit could not remotely prevent the distal end of the flexible spiraled wire from tunneling adjacent to the wall. Such distal extension of the spiraled wire would also increase the force that would have developed between the rotating tip and the wall of the vessel (it would be appreciated by the artisan that much less force has to be applied to an end of a cantilevered beam compared to a force that has to be applied to a middle of a beam, with same cross section, that is supported at both of its ends to cause the same deflection.)

The distal end of the spiraled wire, which forms a part of the distal end of the tip 36 can be smooth. Alternatively the tip can have a tooth with an edge 18 and a face 23 that is positioned to advance towards the obstruction material (please note FIGS. 3 and 9) when the tip is rotated in the second direction 41 to more aggressively starts tunneling whereas when the tip is rotated in the first direction 40 the face 23 continuously retreats from the obstruction material. The distal tip's ability to start tunneling can also be enhanced by forming small teeth like elements on it or bonding sharp particles to it.

To prevent, or to release, fibers and the like from wrapping around the shaft or the tip, the shaft can be rotated back or back and forth in directions 40 and 41. Additionally, sliding the tube 13 back and forth relative to the shaft 14 can be used to dislodge obstruction particles that have formed a clog in the tube or near its distal opening and to enhance the aspiration.

When the vessel is surgically exposed, the rotary catheter can be introduced into the vessel directly through a small puncture in the vessel's wall. However, more commonly, catheters of this type are introduced into the vessel through the skin with the introducer 75 that has a thin-walled plastic sheath 76. As can be appreciated by the artisan, the diameter of the introducer sheath 76 is limited by the size of opening that can be safely punctured in the vessel and making an external diameter 13od of the tube 13 closely fit through the introducer enables the enhancement of the aspiration through the tube as it allows increasing an internal diameter of the tube 13id. It can also be appreciated that enlarging the tip's height 90 to closely fit through the introducer enhances the radial reach 89 of the tip and the size of the tunnel that the tip opens through the obstruction. However, this relationship would cause the tip's height to be larger than the internal diameter 13id of the tube 13 (please note FIG. 8) in which case the tube can be advanced to the tip but not over it. However, where the tip's height is slightly reduced and is smaller than the internal diameter of the tube, the tube can be advanced over the tip to shield it (please note FIGS. 10a, 10b.) In this shielded mode the rotary catheter can readily aspirate soft obstructions that do not have to be broken down prior to entering the tube. The shielded mode of operation is enabled by the tip's flattened shape and reduced cross-sectional area which does not block the tube 13 leaving open aspiration passageways 22′ and 24′ between tip's sides 22 and 24 to the tube's wall, respectively (please note FIG. 10b.) As the clot enters the rotary catheter and gets in between the rotating tip's sides to the tube's wall, whose internal diameter 13id is a bit larger than the tips height 90, the rotating tip macerates the clot so that it can be readily aspirated all the way into the syringe 37. The user can at any time, whether the tip is rotating or not, pull the cylinder 42 and tube 13 to expose the tip and increase its radial reach.

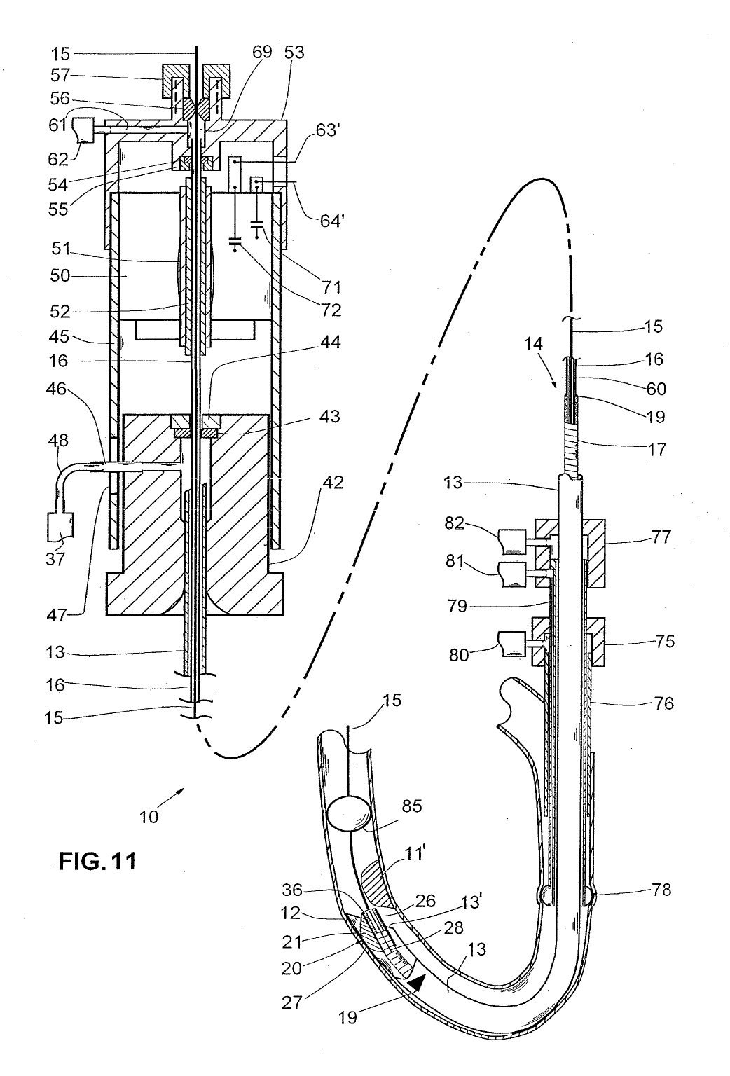

FIG. 11 shows a further modification where the tube 13 is terminated along a diagonal line 13′ so that when the cylinder 42 is partially pulled out of the housing, the tube partially shields the tip. As can be readily understood, the length of the slot 47 can be increased to enable the tube to move from a fully shielding position to a position where the tip and a short section of the spiral are exposed. The configuration shown in FIG. 11 enables the tip to be advanced and urged into contact with an asymmetrical obstruction 11′, which is located on one side of the vessel, while the tube shields an opposite side of the vessel. A radio-opaque marker 19 affixed to the wall of the tube can be used to assist in positioning the tube relative to the obstruction.

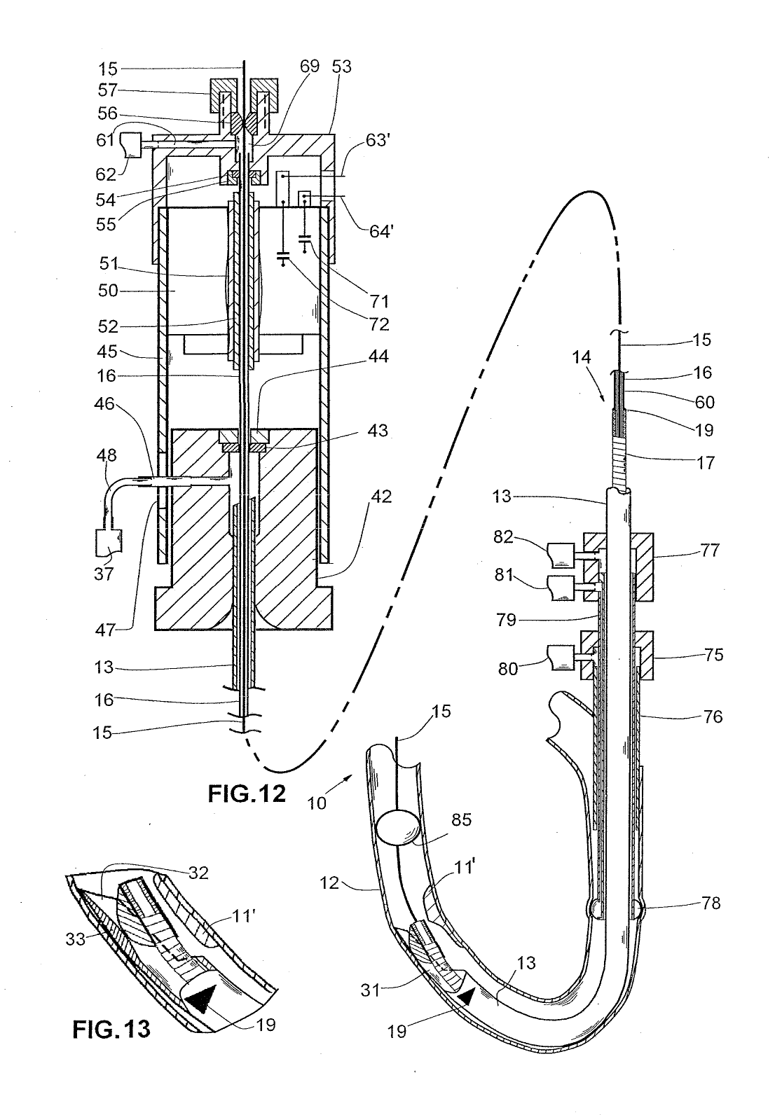

FIG. 12 shows a modification of the rotary catheter of FIG. 11 where a tube's distal end 31 resembles a miniaturized scoop of a gardening trowel. The scoop shields a certain length of one side of the vessel's wall from the rotating tip while urging the rotating tip towards an asymmetrical obstruction 11′ located on the opposite side of the wall. FIG. 13 shows a scoop 32 with a thicker bottom 33 to urge the tip further towards the obstruction. The elongated shape of the scoop 31 allows to protect and to treat a length of the obstruction without having to reposition the scoop in the vessel.

While the present invention has been illustrated with specific embodiment it should be understood that modifications and substitutions may be made within the spirit of the invention and the scope of the claims. For example, the shaft portion 16 can be made to constitute the majority of the length of the shaft 14. Conversely, to enhance the flexibility of the rotary catheter, the portion 17 can be extended to constitute the majority of the length of the shaft 14 to and portion 16 shortened to a point that the connection between the portions 16 and 17 occurs inside the cylinder 42. A further modification is to have the shaft 14 be made of a short proximal tube portion which is connected to a mid spiraled wire portion which is connected to a mid tube portion which is connected to the distal spiraled wire portion. Such a configuration may be useful in a longer rotary catheter needed to reach the heart region from a typical vascular entry point at the groin region. In such an application the mid spiraled wire portion provides enhanced flexibility at the entry region, whereas, the distal spiraled wire portion provides enhanced flexibility needed in the heart region while the mid tube section is sufficiently flexible to be disposed in between these regions (in the relatively straight aorta) while reducing the system's bulk and limiting the elongation of the shaft 14. Because of its simple design the rotary catheter can be made small enough to pass through a guiding catheter (or introducer) having an internal diameter of around 1 millimeters or it can be sealed-up to treat vessels over 10 millimeters in diameter.

1. A rotary catheter for breaking down and for aspirating particles of an obstruction from a bodily vessel, comprising in combination;
a flexible tube containing a motor-driven, flexible, hollow shaft which is rotatable and slideable over a guidewire, at least a distal portion of said shaft being made of a spiraled wire,
a bit affixed to a distal end of said spiraled wire and forming therewith a tip which extends out of said tube distally, said tip having a first side and an opposing second side, said first side adapted to impact said obstruction when said shaft and tip rotate in a first direction,
said tip having a base and an opposing crown, said crown being offset away from a longitudinal axis of said shaft further than said base is offset from said longitudinal axis,
a distance between said sides being smaller than a distance between said crown and said base,
said crown adapted to atraumatically slide against a wall of said vessel and displace a distal end of said tip away from said wall while said tip rotates.

2. As in claim 1 wherein said flexible tube is selectively slideable relative to said shaft.

3. As in claim 2 wherein said tube can be selectively advanced distally, relative to said shaft, to shield said tip.

4. As in claim 1, 2 or 3 wherein suction means are connected to said tube to aspirate fluids and particles of said obstruction through said tube while a relative motion between said tube and said rotating shaft reduces a frictional resistance that said particles encounter while moving through said tube.

5. As in claim 1, 2 or 3 wherein said shaft is rotated in said first direction and a second direction of rotation, back and forth.

6. As in claim 1, 2 or 3 wherein said second side is adapted to impact said obstruction when said shaft is rotated in said second direction.

7. As in claim 6 wherein at least one tooth is located at a distal end of said spiraled wire so that a face of said tooth advances towards said obstruction material when said shaft is rotated in a second direction.

8. As in claim 1, 2 or 3 wherein a flexible guidewire liner is disposed at least in a distal portion of said spiraled wire.

9. As in claim 8 wherein said liner is disposed through the length of said spiraled wire and is mechanically connected to said spiraled wire in a vicinity of said proximal end of said spiraled wire and also in a vicinity of said distal end of said spiraled wire to prevent said liner and spiraled wire from becoming mismatched lengthwise.
